(19) United States
(12) Patent Application Publication
(10) Pub. No.: US 2008/0210393 A1
(43) Pub. Date: Sep. 4, 2008

(54) Mediator-Enzyme System for Controlling Pitch Deposits in Pulp and Paper Production

(75) Inventors: Ana Gutierrez-Suarez, Madrid (ES); Jose Carlos del Rio Andrale, Madrid (ES); Jorge Rencoret Pazo, Madrid (ES); David Ibarra Trejo, Madrid (ES); Ana Mariela Speranza Fernandez, Madrid (ES); Susana Camarero Fernandez, Madrid (ES); Maria Jesus Martinez Hernandez, Madrid (ES); Angel T. Martinez Ferrer, Madrid (ES)

Correspondence Address: NOVOZYMES NORTH AMERICA, INC., 500 FIFTH AVENUE, SUITE 1600, NEW YORK, NY, 10110, US

(73) Assignee: Consejo Superior de Investigacions Cientificas C/ Serrano, 117, Madrid (ES)

(21) Appl. No.: 11/994,685

(22) PCT Filed: Jun. 30, 2006
(86) PCT No.: PCT/ES2006/070091

§ 371 Date: Feb. 14, 2008

(30) Foreign Application Priority Data
Jul. 6, 2005 (ES) P200501648

(51) Int. Cl. D21C 3/00; C12N 9/02

(52) U.S. Cl. 162/72; 435/189

(57) ABSTRACT

The invention relates to the use of mediator-enzyme system, which is characterised in that the enzyme comprises a laccase-type enzyme and the mediator comprises a chemical compound which acts as a redox intermediate in the enzymatic oxidation, for the elimination of lipophilic compounds (including, among others, sterols that are free and conjugated in the form of esters and glycosides, triglycerides, fatty alcohols and resin acids) which cause deposits in the product, the machinery and the circuits during the production of pulp and paper (both from hardwood and softwood and non-woody plants). The treatment eliminates up to 100% of the aforementioned lipophilic compounds, depending on the material treated, and, in this way, improves the quality of the pulp obtained, the operation of the industrial installations and the characteristics of the end product.

### FIELD OF THE INVENTION

The invention is aimed at the industrial sector for pulp and paper manufacturing, including companies that use both non-coniferous and coniferous wood as well as herbaceous or shrub plants as a raw material, and that use both mechanical and chemical manufacturing processes or combinations of both.

### BACKGROUND ART

The manufacturing of paper pulps through the use of less-contaminating technology has brought new problems to pulp manufacturing and bleaching. Among these the formation of so-called pitch deposits (brea or pez in Spanish) occupies an important position as they dramatically reduce the quality of the final product, and even force technical interruptions in the machines, causing serious losses in this industrial sector (Back and Allen. 2000. PITCH control, wood resin and deresination, TAPPI Press, Atlanta). The pitch deposits produced during pulp and paper manufacturing processes are caused by the components of the extractable fraction of the raw material. Other products are often incorporated into said deposits where the extractives act as binding agents.

The components of the extractable fraction of the wood (that which can be obtained using toluene-ethanol, dichloromethane, acetone or other organic solvents) involved in the formation of pitch deposits are generally of a lipophilic nature (soluble in chloroform or hexane) and include fats, waxes, alcohol fats, fatty acids, terpenes, sterols and sterol esters (Hillis; 1962. Wood extractives. Ed: Academic Press, London). The problem with the formation of pitch deposits in conifer pulp manufacturing has been studied extensively due to the fact that this is the type of wood principally used in the main pulp-producing countries (USA, Canada and the Nordic countries of the EU). Among the traditional procedures used for pitch control are found: wood barking, weather-exposed storage of trunks and chips and the addition of agents to the pulps and/or liquids that limit pitch deposits (Allen. 2000. Pitch control in pulp mills. En: Pitch control, Wood resin and deresination, Edited by Back and Allen, TAPPI Press, Atlanta, p. 265-288; and Allen. Pitch control in paper mills. En: Pitch control, Wood resin and deresination, Edited by Back and Allen, TAPPI Press, Atlanta p. 307-328). However, such methods have not been sufficient to eliminate pitch deposit problems in paper pulp.

As an alternative to chemical methods, biological methods have been developed in the last few years for pitch control that are based on the use of micro-organisms or their enzymes (Gutiérrez et al., 2001. Biotechnological control of pitch troubles in paper pulp manufacturing. Trends Biotechnol., 19: 340-348). At present there are commercial preparations for inoculating the wood with micro-organisms (Farrell et al., 1993. Cartapip™: A Biopulping Product for Control of Pitch and Resin Acid Problems in Pulp Mills. J. Biotechnol., 30:115-122; Chen et al., 1994. Wood extractives and pitch problems: Analysis and partial removal by biological treatment. Appita: 47: 463-466) and commercial enzyme complexes with lipase activity for treating paper pulps.

The enzymic methods used to reduce pitch problems during paper pulp manufacturing have focussed mainly on enzymes of the hydrolase type (EC 3.1) including lipases (EC 3.1.1.3) and sterol-esterases (EC 3.1.1.13). Among these it is possible to highlight the recombinant lipase manufactured by Novozymes (formerly a division of Novo Nordisk) and marketed with the name Resinase A®, and the lipases of Candida rugosa and Candida cylindraceae. The results obtained by treating mechanical pine pulp with these lipases shows that the triglycerides are hydrolyzed, the latter being the principal substance responsible for pitch deposits in these pulps (Fujita et al.; 1992. Recent advances in enzymic pitch control. Tappi J., 75: 117-122; Fischer et al.; 1992. Adsorption of lipase on pulp fibers during biological pitch control in paper industry. Enzyme Microb. Technol., 14: 470-473). At present there are several international patent applications that describe the possibility of using lipases to reduce pitch problems (caused by triglycerides) in the pulp and paper industry. In the first, WO9207138, the reduction in content of some lipophilic components of the wood, mainly triglycerides, by adding different lipases during the manufacturing of thermomechanical pulps is described. The second, WO9213130, describes the use of a lipase that resists temperatures of around 70° C. The third, U.S. Pat. No. 5,256,252, describes a method for pitch control with lipase and cationic polymers that capture the fatty acids released by the enzymes. The fourth, WO02055679, describes the reduction of triglycerides by using thermostable variants of a commercial lipase (those in which increases of over 15° C. in thermal stability have been achieved) in the obtainment of mechanical pulp or in the production of paper from this type of pulp.

Despite the fact that pitch problems decrease in conifer pulps, which contain considerable quantities of triglyceride in their extracts (Fengel et al., 1984. Wood: Chemistry, ultrastructure, reactions. Ed: De Gruyter, Berlin; Rowe; 1989. Natural Products of Woody Plants. I and II. Chemicals Extraneous to the Lignocellulosic Cell Wall. Ed: Springer-Verlag, Berlin), treatment with lipase-type enzymes is ineffective in preventing pitch problems in pulps of different conifers and, in particular, in Kraft pulps from Eucalyptus globulus (a species very widely used by the paper industry in Spain and Portugal) and other species of the same genus. In the last few years, research carried out to characterise the extractives of eucalyptus wood has revealed that triglycerides constitute a small fraction of the extractives from Eucalyptus globulus, in which sterols, sterol esters and sterol glucosides have been detected as the principal substances responsible for pitch deposits in the kraft process (del Rio et al., 1998. Characterization of organic deposits in the Kraft pulping of Eucalyptus globulus wood.

J. Chromatogr. 823: 457-465; Gutiérrez et al., 1999. Chemical composition of lipophilic extractives from Eucalyptus globulus Labill. Wood. Hoizforschung, 53. 481-486: Gutiérrez y del Rio 2001. Gas chromatography-mass spectrometry demonstration of sterol glycosides in eucalypt wood, kraft pulp and process liquids. Rapid Commun. Mass Spectrometry; 15: 2515-2520). These compounds remain in the pulp after kraft cooking and TCF (totally chlorine free) bleaching (del Rio et al., 2000. Analysis of pitch deposits produced in Kraft pulp mills using a totally chlorine free bleaching sequence. J. Chromatogr. A; 874: 235-245). Other coniferous species, such as the Populus tremuloides, very widely used in paper manufacturing in North America, also contain notable quantities of free and esterified sterols that cause similar pitch problems (Serreqi et al., 2000. Identification and quantification of important steryl esters in aspen wood. J. Am. Oil Chem. Soc.; 77: 413-418). Moreover, these compounds also form part of conifer wood extractives and reach notable content levels in some of the latter, as is the case with Fices abies wood (Claassen et al. 2000. Rapid Analysis to apolar low molecular weight constituents in wood using high pressure liquid chromatography with evaporative light scattering detection. Phytochem. Anal. 11:251-256; and Serreqi et al.; 2000. Identification and quantification of important steryl esters in aspen wood. J. Am. Oil Chem. Soc.; 77: 413-418). Sterols, sterol esters and sterol glucosides are troublesome due to their resistance to chemical hydrolysis during kraft cooking (compared with triglycerides that are eliminated in the form of fatty acid salts) but they also cause pitch problems during mechanical pulp manufacturing from wood with a high content of these compounds.

There are several patent applications that mention the possibility of using other hydrolase-type enzymes, such as sterol-esterases (EC 3.1.1.13), to eliminate sterol esters during paper pulp manufacturing. The patent WO9423052 suggests the possibility of using an isolated Pseudomonas fragi enzyme in the hydrolysis of lipophilic compounds present in paper pulp, although the conditions in which the pulps must be treated are not described nor are the results after treating the pulp with these enzymes. In the patent WO0053843 the effectiveness of some enzymatic raw materials from fungus and bacteria in the degradation of sterol esters in the treatment of the process liquid of thermomechanical pulp of Picea abies wood is proved and the possibility of using these raw materials in combination with the commercial lipase Resinase A® (that as mentioned previously only hydrolyzes glycerides) is indicated. In this patent the possibility is mentioned of the enzymatic raw materials from fungus and bacteria also being able to have lipase, hemicelullase, pectinase and ‘ligninase’ activity, in addition to sterol-esterase activity, although no test was carried to prove if these activities exist. In the patent WO02075045, a sterol-esterase from the fungus of the Ophiostoma piceae ascomycete type is described that is able to hydrolyze triglyceride and sterol esters simultaneously, and therefore it can be used both in the mechanical processes that use conifers, as well as in the chemical processes that use non-coniferous species. However, the use of sterol-esterases, despite producing hydrolysis of the sterol esters, entails the release of free sterols that are even more detrimental and have a greater tendency to form pitch deposits than the esters due to their stickiness.

There is another patent (WO9216687) that uses an enzymatic method to reduce the pitch problems by means of the use of celullases/hemicelullases. This concerns a method for reducing the pitch deposits associated with mechanical pulps by means of the treatment of the pulp and/or the process liquids with the enzymes mentioned. This process improves some of the pulp's mechanical properties, such as drainage and reduces pitch problems in the pulp and the machinery. However, in this patent details are not given as to whether the compounds responsible for the formation of pitch deposits are eliminated, nor is the mechanism mentioned with which this elimination would be carried out. Patent WO03035972 describes the use of oxygenases (specifically lipoxygenases) that act on unsaturated fatty acids and reduce pitch deposition, probably through peroxidation reactions. However, in this patent only the action of these enzymes on the fatty acids is shown and no indication is given as to whether the other compounds responsible for the formation of pitch deposits mentioned previously (resin acids, fatty alcohol, terpenes and free and conjugated sterols) are eliminated. Finally, patent JP2000080581 describes the use of micro-organisms or plant peroxidases in the presence of hydrogen peroxide and mediators to act on the abietic acid but few details are given on the results obtained.

The enzymatic methods described above have been used with varying success by different pulp-producing companies that use specific processes (fundamentally mechanical) and certain raw materials (fundamentally pine). However, pitch problems have still not been avoided during paper pulp manufacture in the majority of processes (either chemical or mechanical) that use other types of raw materials. This situation has triggered the search for new enzymes that can be used to reduce significantly or eliminate completely pitch problems, the results of which are the object of the present invention.

Laccase-type enzymes (also called phenoloxidases), in the presence of compounds that act as redox mediators, have been described for the bleaching of different types of paper pulp (including patents WO 9429510, WO9501426, WO9954545 and WO03052201) but their use for the elimination of extractible compounds that cause pitch deposits in different paper pulp manufacturing and bleaching processes has not been described. The present study has unexpectedly found that the enzyme-mediator system, that acts by eliminating the compounds derived from lignin that are responsible for pulp colour, also acts with great efficacy on the lipophilic compounds responsible for the formation of pitch deposits. In this way, by using the invention described here, it is possible: i) to eliminate from the pulp and/or process liquids up to 100% of the free sterols, sterol esters, sterol glycosides, triglycerides, fatty acids, resin acids and fatty alcohols (originating from the extractable fraction of the raw material), ii) to reduce the problems of pitch deposits in paper pulps and in machinery caused by these compounds; and iii) to reduce the costs involved in the industrial processes of pulp and paper manufacturing. The enzymatic elimination of the compounds responsible for the formation of pitch deposits present in pulp and process liquids leads to an improvement in the quality of the final product. The enzymatic treatment can reduce, or even eliminate, the number of stoppages due to pitch deposits in the industrial processes of pulp and paper manufacturing, with the consequent saving of time and energy and increase in production. The enzymatic treatment can also improve pulp drainage and, consequently, can lead to the improved behaviour of the pulp in the paper machine. It can also improve the printing characteristics of the paper obtained.

### SUMMARY OF THE INVENTION

The object of the present invention is an enzyme-mediator system where the enzyme is an oxidative enzyme of the laccase group (EC 1.10.3.2) and the mediator is a chemical compound that acts as a redox intermediary in enzymatic oxidation, for the enzymatic control of the lipophilic compounds responsible for the formation of the so-called pitch deposits during paper manufacturing. All or some of the lipophilic compounds on which said enzyme-mediator system acts are from the following compounds: free sterols, conjugated sterols in the form of esters and glycosides, triglycerides, fatty alcohols and resin acids.

The laccase of the enzyme-mediator system can be an enzyme of vegetable, fungal or microbial origin. Appropriate examples of fungal laccases include the laccases of species of Aspergillus, Neurospora (e.g, Crassa Neurospora), Podospora, Botrytis, Collybia, Fomes, Lentinus, Pleurotus, Trametes (e.g, Trametes villosa and Trametes versicolor), Rhizoctonia (e.g, Rhizoctonia solani), Coprinus (e.g, Coprinus cinereus, Coprinus comatus, Coprinus friesii, and Coprinus plicatilis), Psathyrella (e.g. Psathyrella condelleana), Panaeolus (e.g, Panaeolus papilionaceus), Myceliophthora (e.g, Myceliophthora thermophila), Schytalidium (e.g, Scytalidium thermophilum), Polyporus (e.g, Polyporus pinsitus), Phlebia (e.g, Radiata Phlebia), Pycnoporus (e.g. Pycnoporus cinnabarinus) or Coriolus (e.g, Coriolus hirsutus). Appropriate examples of laccases of bacteria include laccases of species of Bacillus.

The preferred laccase-type enzymes are those obtained from fungi cultures of the ligninolytic basidiomycete group, preferably from monocariotic or dicariotic strains from Pycnoporus cinnabarinus or similar species. Said strains from Pycnoporus cinnabarinus are selected for their high capacity to secrete laccase, for example Pycnoporus cinnabarinus CECT 24448 (=IJFM A720, from the Collection of Fungal Cultures of the Centre of Biological Research, Senior Council of Scientific Research, Madrid, Spain). This strain has been filed according to the terms of the Budapest Treaty in the Spanish Type Culture Collection of Microorganisms (CECT), University of Valencia, Edificio de lnvestigación, Campus de Burjasot, E-46100 Burjasot, Valencia, Spain, with the date 31 de October 2001.

Laccase can also be a recombinant enzyme, obtained by means of gene expression (or cDNA) of a laccase of interest using a suitable expression system. The redox mediator of the enzyme-mediator system can be a synthetic compound, preferably 1-hydroxybenzotriazole (HBT) or other compounds of the N—OH type, or a natural mediator, preferably produced by fungi, or a chemical species structurally related to said fungal metabolites. The redox mediator can also be a natural compound formed during the degradation of the lignin, preferably 4-hydroxybenzoic acid, p-coumaric acid, vanillin, syringaldehyde, acetosyringone or different hydroquinones and structurally related chemical species.

Likewise, another object of the present invention consists in a process for the enzymatic control of the lipophilic compounds responsible for the formation of the so-called pitch deposits during paper manufacturing (including free sterols, conjugated sterols in the form of esters and glycosides, triglycerides, fatty alcohols and resin acids), that use the enzyme-mediator system described previously. Said process can be applied to the paper pulp (before or after bleaching) or to the process liquids originating from the cooking, washing or bleaching of the paper pulp that allows up to 100% elimination of said lipophilic compounds, in addition to eliminating undesirable additives, such as anti-foaming agents and recycled paper inks, thus leading to improvements both in the process as well as in the final product.

This process can be used when the paper pulp originates from non-coniferous wood (ligneous angiosperms), including eucalyptus, black poplar, poplar, birch or acacia, namely wood originating from the species Eucalyptus globulus, Eucalyptus camaldulensis, Eucalyptus grandis, Populus tremula, Populus tremuloides, Betula pendula, Betula tremula or Acacia mangium. Likewise, this process can be used when the paper pulp originates from wood from conifers (ligneous gymnosperms), including species of the Picea and Pinus genera, and precisely Picea abies, Pinus sylvestris, Pinus taeda, Pinus contorta o Pinus virginiana. This process can also be used when the paper pulp originates from herbaceous or shrub plants, including agricultural by-products. Said plants can belong both to the dicotyledon group (including, amongst others, linen, hemp, kenaf, cotton and jute) as well as to the monocotyledon group (including, amongst others, sisal, abaca, miscanthus, esparto, wheat, bamboo and bagasse). In the dicotyledon group, amonst others, Linum usitatissimum, (linen), Cannabis saliva (hemp) Corohorus capsularis and Corchorus olitorius (jute) are included and in the monocotyledon group, Agave sisalanae (sisal) and Musa textilis (abaca) are included, amongst others.

The process, object of the present invention, can be used when paper pulp is manufactured by means of so-called mechanical pulping processes, including pulp obtained by means of log milling, by means of chip milling in refiners and by means of thermomechanical methods. In these cases the mechanical pulp is treated with hydrogen peroxide, dithionite or other reactants in order to obtain bleached papers. Said process can also be used when paper pulp is manufactured by means of so-called chemical pulping processes, including kraft cooking, soda cooking (with or without the addition of anthraquinone as a catalyst) or sulphite cooking, and by means of a combination of the so-called chemical and mechanical pulping processes. In these last two cases the paper pulp is bleached by means of processes called TCF (totally chlorine free), ECF (elemental chlorine free) or others.

The paper pulp on which the present process is applied can also be obtained by means of recycled old paper, originating from different original raw materials.

In a particular embodiment of the invention, the enzyme-mediator system is applied during the manufacturing of kraft pulp from Eucalyptus globulus wood bleached by means of TCF or ECF processes.

The application of the enzyme-mediator system used for the enzymatic pitch control is carried out on the pulp at different points of the process, either after cooking, during bleaching or on completion of this. The concentration of laccase applied in this process is comprised between 0.01 and 250 U (=International Units, defined as the quantity of enzyme that oxidizes 1 μmol/min of 2,2′-azino-bis(3-ethylbenzthiazoline-6 sulphonic) acid, ABTS) per gram of pulp, and preferably between 0.01 and 200; 0.01 and 150; 0.01 and 100; or between 0.01 and 50 U/g of pulp. More preferably the laccase is used in a concentration between 0.1 and 100; 0.1 and 75; 0.1 and 50; or between 0.1-25 U/g of pulp. Meanwhile, the mediator is applied at a concentration between 0.1% to 10% (w/w, with respect to pulp dry weight), more preferably in a concentration of 0.1% to 8%; 0.1% to 6%; 0.1% to 5%; 0.1% to 4%; or more preferably at a concentration of 1% to 3% (w/w, with respect to pulp dry weight).

Likewise, the application of the enzyme-mediator system can be carried out on the process liquid originating from the cooking and the washing of the pulp, or the bleaching of the latter. In this case, the laccase can be applied in a free or immobilised form in a suitable support, at a concentration comprised between 0.1-1000 U/l of process liquid, preferably between 0.1 and 800 U/l; between 0.1 and 600 U/l; between 0.1 and 400 U/l; between 0.1 and 200 U/l; or between 0.1 and 100 U/l. The mediator is applied at concentration comprised between 0.1 g/l and 10 g/l, preferably at a concentration of 0.1 to 8, 6, 5 or 4 g/l; more preferably at a concentration of 1 g/l to 3 g/l.

The application temperature of the enzyme-mediator system is comprised between 4° C. and 90° C., between 4° C. and 85, 80, 75, 70 or 65° C.; preferably between 10 and 90° C., between 20 and 90° C., more preferably from 30° C. to 60° C., pH between 3 and 9, and more preferably between 3.5 and 6.5, and with a duration between 10 minutes and 24 hours, and more preferably between 30 minutes and 6 hours.

The laccase-mediator system can also be used for pitch control in combination with other enzymes, more specifically in combination with lipoxygenases that oxidize unsaturated lipids generating lipid radicals, and lipases that hydrolyze triglycerides and other esters of fatty acids.

### BRIEF DESCRIPTION OF THE FIGURES

The following six Figures illustrate the three Examples presented in the following.

FIG. 1. Chromatograms obtained by means of gas chromatography analysis of the silanized lipophilic extracts of a control eucalyptus (Eucalyptus globulus) kraft pulp (A) and of said pulp after treatment with laccase in the presence of the mediator HBT (B).

FIG. 2. Low temperature scanning electron microscopy images (cryo-SEM) of a control eucalyptus (Eucalyptus globulus) kraft pulp (A) and of said pulp after treatment with laccase in the presence of the mediator HBT (B). The bars correspond to 200 μm.

FIG. 3. Fluorescence microscopy images (left) and the corresponding controls in contrast of stages (right) of a control eucalyptus (Eucalyptus globulus) kraft pulp (A and B) and of said pulp after treatment with laccase in the presence of the mediator HBT (C and D).

FIG. 4. Chromatograms obtained after analysis by gas chromatography of the silanized lipophilic extracts of the control pulp of Picea abies (A) and of said pulp after treatment with laccase in the presence of the mediator HBT (B).

FIG. 5. Chromatograms obtained after analysis by gas chromatography of the silanized lipophilic extracts of the control linen pulp (A) and of said pulp after enzymatic treatment with laccase in the presence of the mediator HBT (B).

FIG. 6. Chromatograms obtained by means of gas chromatography analysis of the silanized lipophilic extracts of a eucalyptus (Eucalyptus globulus) kraft pulp after treatment with laccase in the presence of syringaldehyde as mediator and subsequent bleaching with hydrogen peroxide (B) and the corresponding control without laccase or mediator (A).

### DETAILED DESCRIPTION OF THE INVENTION

The object of the present invention is the enzymatic control of pitch deposits by means of the use of oxidative enzymes of the laccase group (EC 1.10.3.2; also called phenoloxidases) in the presence of redox mediators such as 1-hydroxybenzotriazole (HBT) that increase the efficacy of the enzyme during the paper pulp manufacturing processes in which the wood used as a raw material is non-coniferous wood (ligneous angiosperms), in particular species of eucalyptus, black poplar, poplar, birch or acacia and more particularly of Eucalyptus globulus, Eucalyptus camaldulensis, Eucalyptus grandis, Populus tremula, Populus tremuloides, Betula pendula, Betula tremula and Acacia mangium. Likewise, the process of the invention can be applied when the raw material used is coniferous wood (ligneous gymnosperms), in particular from pine or Picea species and more particularly from Pinus sylvestris, Pinus taeda, Pinus contorta, Pinus virginiana, or Picea abies. This invention can also be applied when the raw material used originates from different herbaceous or shrub plants that include species both from the dicotyledon group, amongst others linen (Linum usitatissimum), hemp (Cannabis satir), kenaf (Hibiscus cannabinus), cotton (Gossypium spp) and jute (Corchous spp), as well as those from the monocotyledon group, amongst other sisal (Agave sisalana), miscanthus (Miscanthus sinensis), abaca (Musa textilis), esparto (Stipa tenacissima), curaua (Ananas erectifolius), bamboo (Bambusa sp.) and bagasse (Saccharum officinarum), as well as different agricultural by-products. Likewise, this invention can be applied when the raw material used is old or recycled paper. In this case, in addition to the elimination of the compounds that form the pitch deposits, it will be possible to contribute to the elimination of dyes, additives and other undesirable compounds present in the recycled paper pulp.

The laccase mediator system for pitch control is applicable when paper pulp is manufactured by means of the different pulping processes: mechanical (including log milling, chip milling in refiners and thermomechanical methods), chemical, including kraft cooking, soda cooking (with or without the addition of anthraquinones as a catalyst) and sulphite cooking or combinations of chemical and mechanical methods. It is likewise compatible with several bleaching methods including ECF (elemental chlorine free) and TCF (totally chlorine free) sequences.

In the present invention it is possible to use any enzyme that may be a laccase of vegetable, fungal or microbial origin, specifically a laccase obtained from fungi cultures of the ligninolytic basidiomycete group, and more specifically from monocariotic or dicariotic strains of Pycnoporus cinnabarinus and similar species selected for their high laccase-secreting capacity, including Pycnoporus cinnabarinus CECT 24448 (=IJFM A720) among others. It is also possible to use a recombinant enzyme obtained by means of gene expression (or cDNA) that codifies a laccase of interest using a suitable heterologous expression system.

The enzymatic treatment is carried out in the presence of redox mediators. The mediators to be used during treatment can be synthetic compounds such as HBT or others of N—OH type, or natural compounds synthesized by fungi such as 4-hydroxyanthranilic acid among others, or structurally related chemical species, or natural compounds formed during the degradation of the lignin, such as 4-hydroxybenzoic acid, p-coumaric acid, syringaldehyde, vanillin, acetosyringone, or different hydroquinones and structurally related chemical species.

The treatment with the laccase-mediator system is applied to the pulp after cooking, during bleaching, or on completion of this, at a temperature comprised between 4° C. and 90° C., preferably between 30° C. and 60° C., and at a pH between 3 and 9, preferably between 3.5 and 6.5 for a time between 10 minutes and 24 hours, preferably between 30 minutes and 6 hours. The concentration of laccase is comprised between 0.01 and 250 U/g of pulp, preferably between 0.1 and 25 U/g of pulp, and the concentration of mediator is comprised between 0.1° 1% and 10% (referring to the dry weight of the pulp), preferably between 1% and 3%, and achieves an elimination of lipophilic compounds, estimated by gas chromatography with a flame detector and gas chromatography coupled with mass spectrometry, of up to 100% of free sterols, sterol esters, sterol glucosides, resin acids, triglycerides and other lipophilic extractives. Other preferred conditions are those mentioned above (in the section of the summary of the invention). The units and dosage are also those defined previously.

When the process is applied to the process liquids originating from the cooking and the washing of the pulp or the bleaching of the latter, the enzymatic treatment is also applied at a temperature comprised between 4° C. and 90° C., preferably between 30° C. and 60° C., and at a pH between 3 and 9, preferably between 3.5 and 6.5 for a time period between 10 minutes and 24 hours, preferably between 30 minutes and 6 hours. The concentration of laccase is comprised between 0.1-1000 U/l of process liquid, preferably with the immobilised enzyme on a suitable support, and the concentration of mediator is comprised between 0.1-10 g/l, preferably between 1-3 g/l, and achieves an elimination of the sterols, sterol esters, sterol glucosides, resin acids, triglycerides and other lipophilic extractives of up to 100%. Other preferred conditions are those mentioned above in the section of summary of the invention.

The percentage of enzymatic elimination of lipophilic compounds is determined by gas chromatography of the extracts of the treated pulp and the treatment liquids. This process includes the following stages: i) the pulp treated with enzyme-mediator (and an untreated sample, as a control) is filtered, dried and extracted with acetone in a Soxhlet for 8 hrs; ii) the filtration liquids are extracted in a separating funnel with methyl-tert-butyl ether; iii) all the extracts are evaporated to dryness and are re-dissolved in chloroform for the analysis of the lipophilic fraction by gas chromatography and gas chromatography/mass spectrometry, after derivatization with bis(trimethylsilyl)trifluoroacetamide (BSTFA) when necessary.

### EMBODIMENTS OF THE INVENTION

The present invention is additionally illustrated by means of the following examples, which are not intended to be limitative to its scope.

### Example 1

### Treatment of Eucalyptus Kraft Pulp with Laccase (from Pycnoporus cinnabarinus) and HBT (1-hydroxybenzotriazole)

To check the effectiveness of the treatment of the eucalyptus pulp with the laccase-mediator system in the elimination of the compounds responsible for the formation of pitch deposits, eucalyptus (Eucalyptus globulus) kraft pulp is used that contains free sterols, sterol esters and sterol glucosides.

The laccase used in this and in the following examples was obtained in 1 m³ digesters with cultures from a Pycnoporus cinnabarinus strain that produces laccase, such as CECT 24448 (=IJFM A720), cultivated according to that described by Herpoël et al. (Herpoël et al. 2000, Selection of Pycnoporus cinnabarinus strain for laccase production, FEM Microbiol. Lett. 183: 301-306). A unit of laccase activity was defined as the quantity of enzyme that oxidizes one μmol/min of 2,2′-azino-bis(3-ethylbenzthiazoline-6-sulphonic acid (ABTS) to the corresponding cationic radical, at 24° C.

We started with an initial raw pulp with a kappa index of 14. This pulp was subjected to a double oxygen stage where a pressure of 6 kg/cm² of oxygen in alkaline conditions was used. This pulp was then subjected to the treatment stage with laccase-mediator. For this, 200 g (dry weight) of pulp delignified with oxygen, at a consistence of 10%, was treated in a 4-litre reactor with 20 U of laccase/g of pulp, 4000 U/reactor (82 ml of laccase with activity of 50 U/ml of raw material) at 50° C. and pH 4, for 120 minutes in the presence of HBT at 1.5% (p/p), with agitation for 1 minute every 10 minutes (at 60 rpm). To identify more effectively the results of the treatment with laccase-mediator, a second sequence was applied including a control stage in the same conditions but without the addition of laccase or mediator (stage called “a” as it was carried out in weak acidic conditions, at pH 4).

The enzymatically treated pulps as well as the corresponding controls were filtered, dried and extracted in Soxhlet with acetone for 8 hours. Likewise, the liquids obtained after filtering of the enzymatically treated pulp as well as the corresponding controls were extracted with methyl-tert-butyl-ether. The extractable fraction of pulps and liquids was dried with nitrogen and the compounds soluble in chloroform were analysed and quantified by gas chromatography and gas chromatography coupled with mass spectrometry following derivatization with bis(trimethylsilyl)trifluoroacetamide (BSTFA) in the presence of pyridine. The lipophilic compounds were analysed in an Agilent Technologies 6890N Network GC system chromatograph, equipped with a flame detector and a silica capillary column of 5 m×0.25 mm and 0.1 mm film thickness (DB-5HT of J & W Scientific) using helium as a carrier gas. The injector and the detector temperatures were 300° C. and 350° C. respectively. The oven was programmed from 10° C. (1 minute) to 350° C. (3 minutes) at 15° C./minute. The gas chromatography-mass spectrometry analysis was carried out in Varian Saturn 2000 equipment with an ion trap detector, using a silica capillary column of 12 m×0.25 mm and 0.1 mm film thickness PB-5HT of J & W Scientific) and using helium as a carrier gas. The different compounds were quantified using a calibration curve carried out with mixes of standards (the correlation coefficients were greater than 99%).

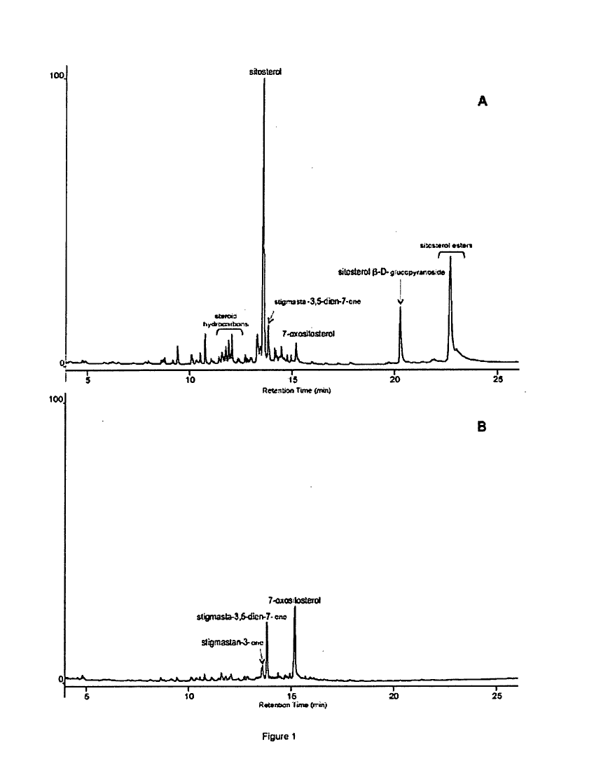

FIG. 1 shows the analysis by gas chromatography of the lipofilic extract of the control eucalyptus kraft pulp (A) and of the extract of eucalyptus kraft pulp treated with laccase in the presence of HBT (B). Said analysis shows that the treatment with laccase-HBT eliminates completely both the free sterols as well as the sterol esters and sterol glucosides present in the eucalyptus pulp. These compounds are the substances mainly responsible for pitch deposit formation during the paper pulp manufacturing process from eucalyptus wood and are found in large proportions in raw pulps and pulps bleached with TCF sequences.

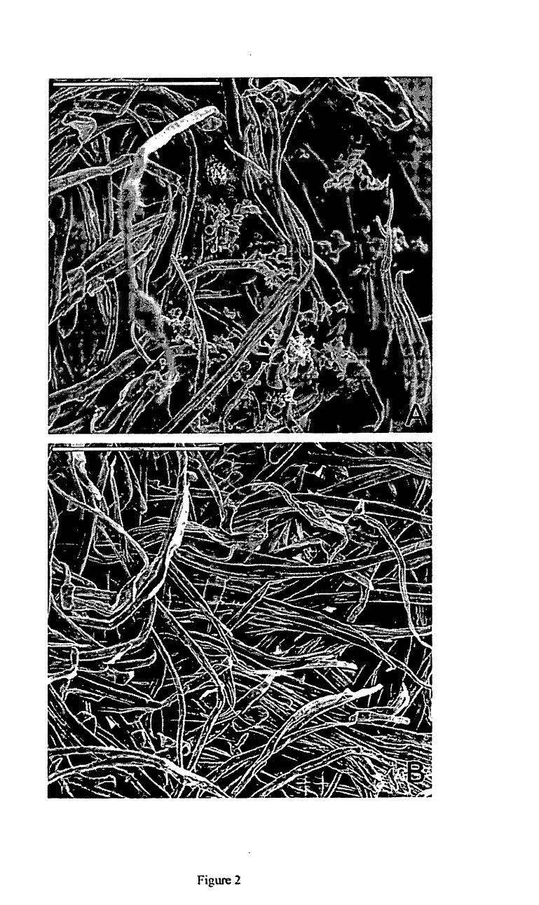

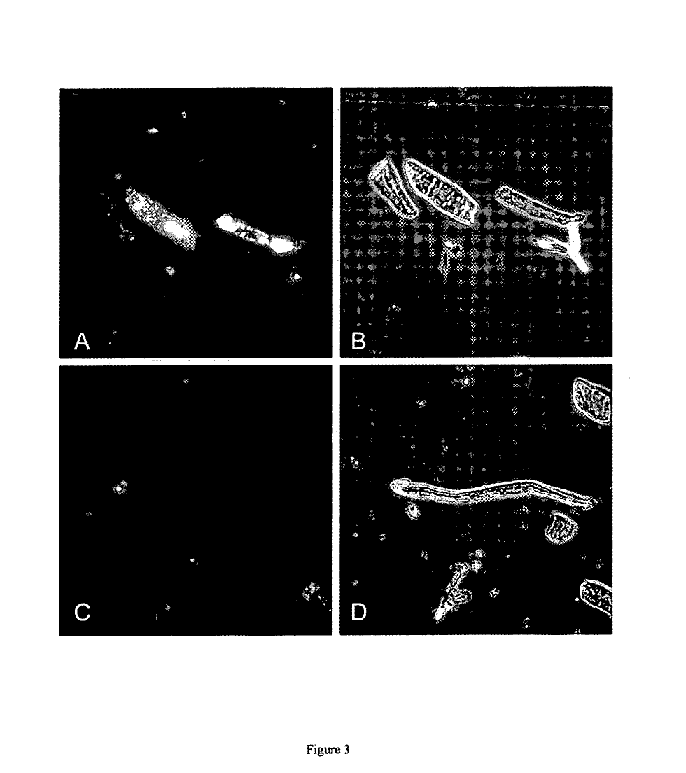

The treated pulp and the control pulp were examined by means of low temperature scanning electron microscopy as well as by means of fluorescence microscopy after filipin staining that reacts specifically with the sitosterol forming fluorescent complexes. In the first case the disappearance of the material deposited between the fibres (a part of which probably corresponds to pitch deposits) after treatment with the laccase-mediator system (FIG. 2) was observed. In the second case, the staining of the sitosterol with filipin showed a notable elimination from this compound (particularly abundant in cells of the parenchymatic radii present in the pulp) after the treatment with the laccase-mediator system (FIG. 3).

### Example 2

### Treatment of Picea Thermomechanical Pulp with Laccase (from Pycnoporus cinnabarinus) and HBT (1-hydroxybenzotriazole)

To check the effectiveness of the treatment with laccase-mediator for the elimination of the compounds responsible for the formation of pitch deposits in the manufacturing of Picea pulp, treatment was carried out on Picea abies thermomechanical pulp, with a high content in triglicerides, sterol esters, resin acids and fatty acids.

10 g of raw Picea pulp (obtained after the first refining) with a consistence of 10% was treated for 2 hours at 50° C. with 20 U of laccase/g of pulp in the presence of the mediator HBT at 1.5% (w/w) using 50 mM tartrate buffer, pH 4, under a humid current of oxygen. Simultaneously, controls were carried out without enzymes in the same conditions. The pulp treated enzymatically as well as the controls were dried and extracted in Soxhlet with acetone for 8 hours. The extractable fraction was dried and the compounds soluble in chloroform were analysed and quantified by gas chromatography and gas chromatography coupled with mass spectrometry according to the conditions detailed in Example 1.

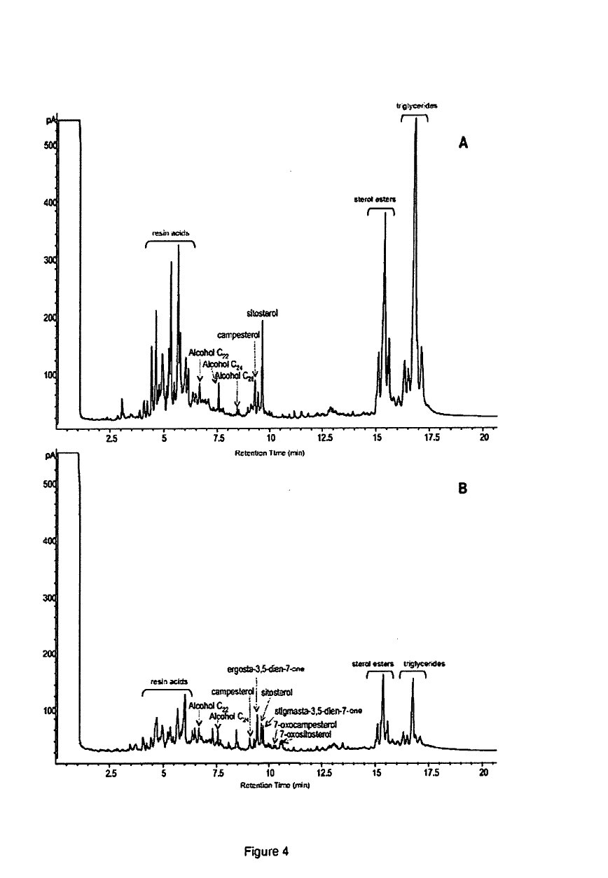

FIG. 4 show the chromatograms obtained after analysis by gas chromatography of the lipophilic extracts of the Picea abies thermomechanic control pulp (A) and of said pulp after treatment in the presence of laccase HBT (B). Said analysis shows that the treatment of the Picea thermomechanical pulp with laccase-HBT reduces triglycerides (by 90%), sterol esters (by 80%), free sterols (by 90%), resin acids and fatty acids (by 90%) present in the Picea pulp. These compounds are the substances mainly responsible for the formation of pitch deposits during the manufacturing process of paper pulp from Picea wood.

### Example 3

### Treatment of Linen Soda-Anthraquinone Pulp with Laccase (from Pycnoporus cinnabarinus) and HBT (1-hydroxybenzotriazole)

To check the effectiveness of the treatment of the linen pulp with the laccase-mediator system in the elimination of the compounds responsible for the formation of pitch deposits, soda-anthraquinone pulp of Linum usitatissimum that contains fatty alcohols, free sterols, and sterol glucosides was used.

40 g of raw linen pulp (kappa index 11) was treated at 3% consistence for 24 hours at 30° C. with 20 U of laccase/g of pulp in the presence of the mediator HBT at 3% (w/w) in 50 mM tartrate buffer, pH 4, and in the presence of Tween 80 at 0.05% in a reactor with pressurised oxygen (6 bar). The pulps treated enzymatically, as well as the corresponding controls were dried and extracted in Soxhlet with acetone for 8 hours. The extractable fraction was dried and the compounds soluble in chloroform were analysed and quantified by gas chromatography and gas chromatography coupled with mass spectrometry according to the conditions detailed in Example 1.

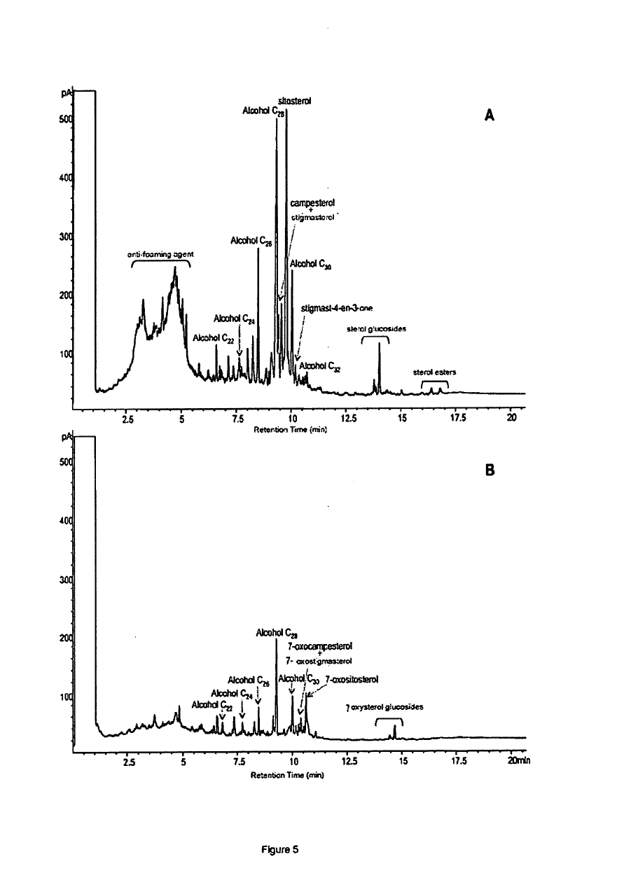

FIG. 5 shows the analysis by gas chromatography of the lipophilic extract of the control linen pulp (A) and of said pulp after enzymatic treatment with laccase in the presence of HBT (B). Said sample shows that the treatment of the linen soda-anthraquinone pulp with laccase-HBT reduces the fatty alcohol (by 80%), free sterols (by 100%) and sterol glucosides (by 90%) present in said pulp. These compounds are the substances mainly responsible for the formation of pitch deposits during the manufacturing process of paper pulp from linen fibre. It is possible to observe how the anti-foaming agent that appears in the control pulp and that also causes of the formation of pitch deposits, is almost completely eliminated after enzymatic treatment with laccase-HBT.

### Example 4

### Treatment of Eucalyptus Kraft Pulp with Laccase (from Pycnoporus cinnabarinus) and Syringaldehyde (4-hydroxy-3,5-dimethoxybenzaldehyde)

To check the effectiveness of the laccase-mediator system, using a natural compound (formed during lignin degradation) such as redox mediator, in the elimination of the compounds responsible for the formation of pitch deposits during the manufacturing of eucalyptus pulp, pulp kraft from Eucalyptus globulus is used that contains free sterols, sterol esters and sterol glucosides We started with an initial raw pulp with a kappa index of 15.8. This pulp (10 g) was subjected to treatment with laccase-mediator at atmospheric pressure with oxygen bubbling. The treatment is carried out using laccase (20 U of laccase/g of pulp) in the presence of syringaldehyde (6.75 mM) as mediator, at pH 4, for 12 hours at 50° C., at a consistence of 3% and with agitation at 170 rpm. The pulp treated enzymatically was subsequently subjected to a bleaching stage with hydrogen peroxide in alkaline conditions. Simultaneously, controls were carried out without enzymes or mediators in the same conditions.

The pulps treated enzymatically and these same pulps after bleaching with hydrogen peroxide, as well as the corresponding controls were filtered, dried and extracted in Soxhlet with acetone for 8 hours. Likewise, the liquids obtained after filtering of the pulp treated enzymatically as well as the corresponding controls were extracted with methyl-tert-butyl-ether. The extractable fraction of pulps and liquids was dried with nitrogen and the compounds soluble in chloroform were analysed and quantified by gas chromatography and gas chromatography coupled with mass spectrometry according to the conditions detailed in Example 1.

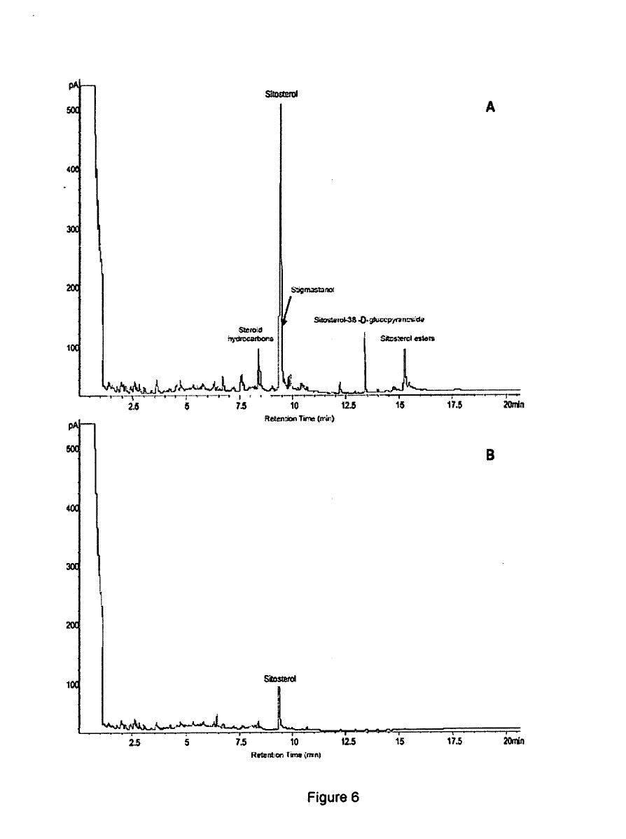

FIG. 6 shows the analysis by gas chromatography of the lipophilic extract of the control pulp (eucalyptus kraft pulp) after bleaching with hydrogen peroxide (A) and of the eucalyptus kraft pulp extract treated enzymatically (with laccase in the presence of syringaldehyde) after bleaching with hydrogen peroxide (B). Said analysis shows that the treatment with laccase-syringaldehyde (followed by a peroxide stage) completely eliminates both the free sterols (91%) as well as the sterol esters (100%) and sterol glucosides (93%) present in the eucalyptus pulp. These compounds are the substances mainly responsible for the formation of pitch deposits during the manufacturing process of paper pulp from eucalyptus wood and are found in large proportions in raw pulps and pulps bleached with TCF sequences.

1-43. (canceled)

44. A composition comprising an oxidative enzyme of the laccase group (EC 1.10.3.2) and a mediator which is a chemical compound that acts as an intermediary redox in enzymatic oxidation for the enzymatic control of the lipophilic compounds responsible for the formation of the so-called pitch deposits during paper pulp manufacturing.

45. The composition of claim 44, wherein the lipophilic compounds on which the enzyme acts can be extracted with organic solvents such as toluene-ethanol, dichloromethane and acetone.

46. The composition of claim 45, wherein the extractable lipophilic compounds on which the enzyme acts are all or partly selected from the group consisting of free sterols, sterols conjugated in the form of esters and glycosides, triglycerides, fatty alcohols and resin acids.

47. The composition of claim 46, wherein the sterols on which the enzyme acts are free sitosterol, sitosterol esters, such as sitosterol oleate or linoleate, or sitosteryl 3β-D-glucopyranoside.

48. The composition of claim 44, wherein the laccase is obtained from fungi cultures from the lignolitic basidiomycete group.

49. The composition of claim 48, wherein the laccase is obtained from monocariotic or dicariotic strains from Pycnoporus cinnabarinus or similar species.

50. The composition of claim 49, wherein the laccase is obtained from Pycnoporus cinnabarinus CECT 24448.

51. The composition of claim 44, wherein the redox mediator is 1-hydroxybenzotriazole (HBT) or other compounds of the N—OH type.

52. The composition of claim 44, wherein the redox mediator is selected from the group consisting of 4-hydroxybenzoic acid, p-coumaric acid, syringaldehyde, vanillin, acetosyringone, or different hydroquinones and structurally related chemical species.

53. A process for the enzymatic control of the lipophilic compounds responsible for the formation of the so-called pitch deposits during paper pulp manufacturing, comprising treating the lipophilic compounds with a composition of claim 44.

54. The process of claim 53, wherein the composition is applied on the paper pulp (before or after bleaching) or on the process liquids originating from the cooking, washing or bleaching of the paper pulp.

55. The process of claim 53, wherein at least 20% of the lipophilic compounds responsible for the formation of the so-called pitch deposits are eliminated from the paper pulp or from the liquids produced as a result of the process.

56. The process of claim 53, wherein the lipophilic compounds responsible for the formation of the so-called pitch deposits are lipophilic compounds that can be extracted with organic solvents.

57. The process of claim 53, wherein the wood used as raw material for paper pulp manufacturing is non-coniferous wood (ligneous angiosperms).

58. The process of claim 53, wherein the wood used as raw material for paper pulp manufacturing is wood from conifers (ligneous gymnosperms).

59. The process of claim 53, wherein the raw material used for paper pulp manufacturing originates from herbaceous or shrub plants including agricultural by-products.

60. The process of claim 53, wherein the composition is applied to the pulp after cooking, during bleaching or on completion of the latter.

61. The process of claim 53, wherein the enzyme is applied at a concentration comprised between 0.01 and 250 U/g of pulp.

62. The process of claim 53, wherein the redox mediators are applied at a concentration of 0.1% to 10% (with respect to pulp dry weight).

63. The process of claim 53, wherein the composition is applied to the process liquids originating from the cooking and the washing of the pulp or the bleaching of the latter.
